(19) United States
(12) Patent Application Publication
(10) Pub. No.: US 2025/0295416 A1
(43) Pub. Date: Sep. 25, 2025

(54) MEDICAL SYSTEMS, DEVICES, AND METHODS FOR DELIVERING ONE OR MORE TREATMENT AGENTS

(71) Applicants: Boston Scientific Scimed, Inc., Maple Grove, MN (US); Boston Scientific Medical Device Limited, Galway (IE)

(72) Inventors: Deepak Kumar SHARMA, Muzaffarnagar (IN); Charudatta Chandrakant ARADHYE, Solapur (IN); Kevin L. BAGLEY, Natick, MA (US)

(73) Assignees: Boston Scientific Scimed, Inc., Maple Grove, MN (US); Boston Scientific Medical Device Limited, Galway (IE)

(21) Appl. No.: 19/082,764

(22) Filed: Mar. 18, 2025

Related U.S. Application Data
(60) Provisional application No. 63/568,091, filed on Mar. 21, 2024.

(51) Int. Cl. A61B 17/12; A61B 17/00

(52) U.S. Cl. CPC A61B 17/12186; A61B 17/00491; A61B 2017/00867; A61B 2017/12004; A61B 2017/1205

(57) ABSTRACT

Disclosed is a medical device comprising: a handle; a shaft extending from a distal end of the handle, the shaft defining a lumen; a pull wire disposed in the lumen of the shaft; and an agent delivery member connected to a distal end of the pull. The agent delivery member may comprise an expandable member and an absorbent member. The expandable member may be configured to transition from a collapsed configuration to an expanded configuration, and the expandable member may be biased to the expanded configuration.

### CROSS-REFERENCE TO RELATED APPLICATIONS

This application claims the benefit of priority to U.S. Provisional Application No. 63/568,091, filed on Mar. 21, 2024, which is incorporated by reference herein in its entirety.

### TECHNICAL FIELD

The disclosure relates generally to systems, devices, and methods for delivering one or more treatment agents. More specifically, aspects of the disclosure pertain to systems, devices, and/or methods for delivering one or more treatment agents via an agent delivery device provided in a medical device, such as an endoscope.

### BACKGROUND

For endoscopic procedures, treatment agents, such as adhesives or other hemostatic agents, may be used to promote natural healing by creating a protective layer that minimizes delayed bleeds, potential perforation, and stricture formation. These adhesives may be, for example, a viscous gel. Endoscopically delivering a viscous agent may be difficult as a large amount of force is necessary to deliver the agent. Insufficient or incomplete delivery of the agent may increase the risks, costs, and duration of a medical procedure.

The systems, devices, and methods of this disclosure are provided to rectify some of the deficiencies described above or to address other aspects of the art.

### SUMMARY

This disclosure includes medical systems and devices for delivering one or more treatment agents (e.g., a biocompatible adhesive) and methods of use thereof, e.g., methods of delivering one or more treatment agents to a target site of a patient, for example, to help heal an ulcer and/or to perform hemostasis.

In an example, a medical device may comprise: a handle; a shaft extending from a distal end of the handle, the shaft defining a lumen; a pull wire disposed in the lumen of the shaft; and an agent delivery member connected to a distal end of the pull. The agent delivery member may comprise an expandable member and an absorbent member. The expandable member may be configured to transition from a collapsed configuration to an expanded configuration, and the expandable member may be biased to the expanded configuration.

Any of the aspects disclosed herein may include any of the following features, alone or in combination. The lumen may be configured to transmit a fluid from the handle of the device to the agent delivery member. The absorbent member may include a powdered treatment agent. The powdered treatment agent is configured to mix with a fluid delivered through the lumen to form a viscous fluid treatment gel. In the expanded configuration, the agent delivery member may have a curved cone shape. The expandable member may include a memory shape element, The expandable member may include nitinol. The expandable member includes braided nitinol wires. The expandable member may be formed of a plurality of discrete wires that are biased to a flared shape in the expanded configuration. Each of the plurality of wires may include an absorbent member. The expandable member may be configured to expand radially outward when the agent delivery member is deployed from the distal end of the shaft. The absorbent member may include a sponge. The pull wire may define a pull wire lumen. The pull wire lumen may be configured to transmit a fluid to the absorbent member. The absorbent member may be on a radially inner surface of the expandable member.

In another aspect, a medical device may comprise: a handle; a shaft extending from a distal end of the handle, the shaft defining a lumen; a pull wire connected to the handle; and an agent delivery member connected to a distal end of the pull wire, the agent delivery member comprising an absorbent member. The shaft may include a lumen for delivering fluid to the absorbent member.

Any of the aspects disclosed herein may include any of the following features, alone or in combination. The lumen for delivering fluid to the absorbent member may be disposed in the pull wire.

In another aspect, a method of using a medical device may comprise: delivering fluid to the absorbent member of the agent delivery member; moving the agent delivery member distally so that it transitions from a collapsed configuration to an expanded configuration; and using the agent delivery member to apply an agent to a bodily lumen. The medical device may comprise an agent delivery member connected to a distal end of a pull wire. The agent delivery member may comprise an expandable member and an absorbent member.

Any of the aspects disclosed herein may include any of the following features, alone or in combination. The absorbent member may include a powdered treatment agent configured to mix with the fluid to form a viscous fluid treatment gel. The expandable member may include a shape memory element. In the expanded configuration, the agent delivery member may have a curved cone shape.

### BRIEF DESCRIPTION OF THE DRAWINGS

The accompanying drawings, which are incorporated in and constitute a part of this specification, illustrate examples of this disclosure and together with the description, serve to explain the principles of the disclosure.

FIG. 1A depicts a perspective view of a proximal portion of an exemplary medical system according to one or more aspects.

FIG. 1B depicts a perspective view of a distal portion of the exemplary medical system, according to one or more aspects.

FIG. 1C depicts an enlarged view of the distal portion of FIG. 1B.

FIG. 2A depicts a perspective view of a distal portion of the exemplary medical system with an agent delivery member deployed, according to one or more aspects.

FIG. 2B depicts a perspective view of an expandable member, according to one or more aspects.

FIG. 3 depicts a perspective view of a distal portion of the exemplary medical system with an agent delivery member deployed, according to one or more aspects.

FIG. 4A depicts a cross-sectional view of a distal portion of the exemplary medical system within a bodily lumen, according to one or more aspects.

FIG. 4B depicts a cross-sectional view of a distal portion of the exemplary medical system within a bodily lumen with an agent delivery member deployed, according to one or more aspects.

FIG. 5A depicts a cross-sectional view of a distal portion of the exemplary medical system within a bodily lumen, according to one or more aspects.

FIG. 5B depicts a cross-sectional view of a distal portion of the exemplary medical system within a bodily lumen with a fluid being applied to the agent delivery member, according to one or more aspects.

FIG. 5C depicts a cross-sectional view of a distal portion of the exemplary medical system within a bodily lumen with an agent delivery member deployed, according to one or more aspects.

### DETAILED DESCRIPTION

To endoscopically deliver a treatment agent to target areas, such as in the esophagus, stomach, intestine, and colon, the following systems, devices, and methods are disclosed. Treatment agents, such as adhesive agents (e.g., prophylactic adhesive agents) may be viscous and thus difficult to deliver. An agent delivery device, including a delivery member may be used in conjunction with other elements of a medical system to deliver the prophylactic treatment agent.

Reference is now made in detail to examples of this disclosure, aspects of which are illustrated in the accompanying drawings. The terms “proximal” and “distal” are used herein to refer to the relative positions of the components of an exemplary medical system and exemplary medical devices. When used herein, “proximal” refers to a position relatively closer to the exterior of the body of a subject or closer to a medical professional using the medical system or medical device. In contrast, “distal” refers to a position relatively further away from the medical professional using the medical system or medical device, or closer to the interior of the body of the subject. Proximal and distal directions are labeled with arrows marked “P” and “D”, respectively, throughout various figures. As used herein, the terms “comprises,” “comprising,” “has,” “having,” “includes,” “including,” or other variations thereof, are intended to cover a non-exclusive inclusion, such that a system, device, or method that comprises a list of elements does not include only those elements, but may include other elements not expressly listed or inherent thereto. Unless stated otherwise, the term “exemplary” is used in the sense of “example” rather than “ideal.” As used herein, the terms “about,” “substantially,” and “approximately,” indicate a range of values within +/−10% of a stated value. Wherever possible, the same reference numbers will be used throughout the drawings to refer to the same or like parts. Unless otherwise specified, the features of the following embodiments maybe combined together in any combination or subcombination.

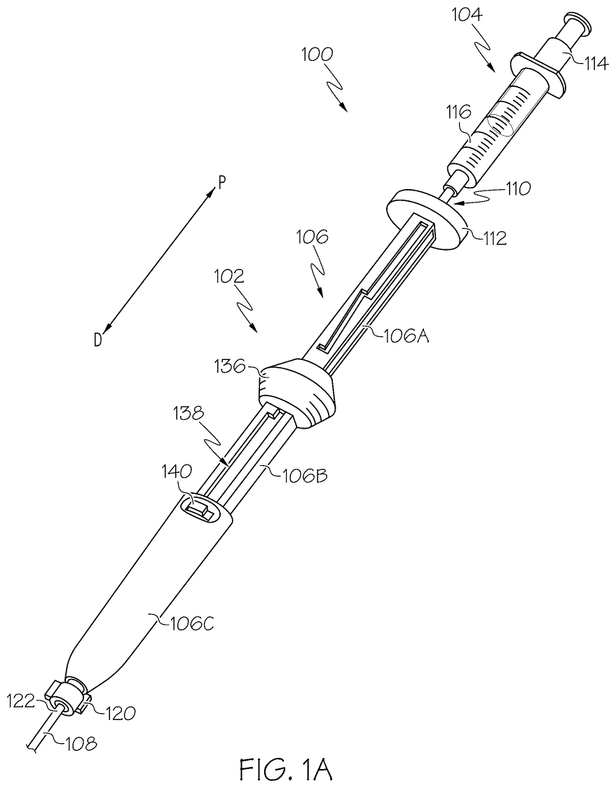

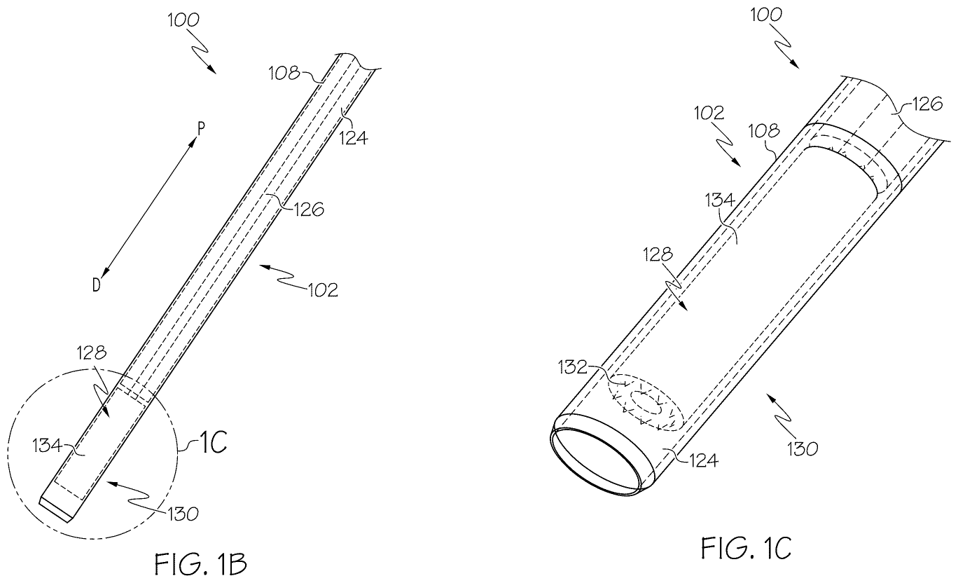

FIG. 1A illustrates an exemplary medical system 100, including a first medical device 102 and a second medical device 104. Although not depicted in all figures, the second medical device 104 may be used with any of the aspects disclosed herein. The first medical device 102 may be an insertion device, and may include a handle 106 and an insertion portion 108 (e.g., a shaft or a sheath). The second medical device 104 may be a fluid delivery device or reservoir, such as a syringe. The first medical device 102 may include a first port 110 disposed on a proximal end 112 of the handle 106 for receiving the second medical device 104 such that a fluid that is stored in the second medical device 104 may be transferred to the first medical device 102 via an actuation of the second medical device 104. For example, the actuation of the second medical device may be performed by pushing a plunger 114 of a second medical device 104 to transmit a fluid from with a cylinder 116 (e.g., barrel) of the syringe through the first port 110 to the first medical device 102. The connection between the first medical device 102 and the first port 110 may include a luer lock connection (or any other suitable type of connection) to provide a leak-proof seal at the first port 110.

The first port 110 may be connected to a hollow interior (e.g., a lumen) of the handle 106 that extends from the proximal end 112 of the handle 106 to a distal end 120 of the handle 106. Fluid is transferred from the second medical device 104 through the first port 110 to the hollow interior. At the distal end 120 of the handle 106, a second port 122 may be provided, fluidly connecting the hollow interior to the insertion portion 108. The insertion portion 108 may be a shaft with a hollow lumen 124, as shown in FIG. 1B. The hollow interior of the handle 106 may be in fluid communication with the lumen 124, such that fluid (e.g., an agent) may pass from through the hollow interior of the handle 106 and into the lumen 124.

FIG. 1B depicts a perspective view of the distal portion of insertion portion 108 of the first medical device 102, according to one or more aspects. FIG. 1C depicts an enlarged view of a portion of the distal portion of FIG. 1B. Broken lines are used to depict elements that are internal of other structures in FIGS. 1B and 1C and in other figures herein. An agent delivery member 128 may be positioned at or near the distal end 130 of the insertion portion 108, within the lumen 124. In FIG. 1B, the agent delivery member 128 may have a collapsed configuration. The agent delivery member 128 may include an expandable member 132 (e.g., a stent, frame, scaffold, or other structure) and an absorbent member 134. In some aspects, the expandable member 132 may incorporate resilient shape memory elements, such as nitinol (nickel titanium alloy) wires, nitinol wings, and/or other components formed from nitinol or other materials with shape memory properties. In some aspects, the absorbent member 134 may be a foam or a sponge, or another porous body.

In examples, absorbent member 134 may be positioned on an inner surface and/or an outer surface of expandable member 132. In some examples, expandable member 132 may be embedded within absorbent member 134. Absorbent member 134 may cover an entirety or only a portion of expandable member 132.

In some aspects, the first medical device 102 may be inserted into a medical scope, such as gastroscope. Although a gastroscope is referenced herein, such reference is not limiting, and first medical device 102 may be used in conjunction with other types of medical devices, such as, for example, endoscopes, ureteroscopes, duodenoscopes, endoscopic ultrasonography (“EUS”) scopes, colonoscopes, bronchoscopes, laparoscopes, arthroscopes, cystoscopes, aspiration scopes, sheaths, catheters, etc. The first medical device 102 may be inserted through the working channel of the medical scope and extended from a distal opening of the working channel, such that the first medical device 102 is positioned at a target site within a body lumen of a subject.

In some aspects, the second medical device 104 includes a treatment agent, such as a prophylactic treatment agent or another type of hemostatic agent. As discussed above, the treatment agent may be delivered into the lumen 124 of the insertion portion 108. In other examples, the treatment agent may be pre-loaded into the lumen 124 before a procedure. Thus, the treatment agent may contact, penetrate, and/or saturate absorbent member 134. It may be difficult to transport an agent delivery member through a narrow opening of a medical device, such as the shaft of the first medical device 104. To overcome this difficulty, in some aspects, a dry agent delivery member 128 is disposed at or near the distal end 130 of the insertion portion 108 and fluid is applied thereto when it is ready for deployment from the hollow interior of the insertion portion 108. The agent delivery member 128 may be positioned into place at or near the distal end of the shaft via manipulation of the control wire 126, which, as discussed above, may extend from the handle 106 through the hollow interior of the insertion portion 108.

With to FIGS. 1A and 1B, the handle 106 may include a spool 136 and a ratchet 138. A control wire 126 may extend from the spool 136 of handle 106, through the lumen 124, to the agent delivery member 128. A distal end of control wire 126 may be coupled to the agent delivery member 128, such that movement of the control wire 126 moves the agent delivery member 128.

The control wire 126 may be connected to the spool 136 such that a practitioner is able to move the control wire 126 in a longitudinal direction (coaxial with or parallel to a central longitudinal axis of the insertion portion 108) by actuating the spool 136 (e.g., by moving the spool 136 distally). For example, the control wire 126 may be moved in a distal direction D by pushing the spool 136 in a distal direction D, and/or the control wire 126 may be moved in a proximal direction P by pushing the spool 136 in a proximal direction P. The spool 136 may be fixedly coupled to a first portion 106A of the handle 106 (e.g., a proximal portion of the handle 106). The first portion 106A of the handle 106 may include the proximal end 112 and a shaft 106B.

The first portion 106A of the handle 106 may be movable relative to a second portion 106C (e.g., a distal portion) of the handle 106. For example, the second portion 106C of the handle may define a cavity, in which the first portion 106A of the handle 106 is movably (e.g., slidably) received. For example, a practitioner may grip or otherwise hold the spool 136 in order to move the first portion 106A of the handle relative to the second portion 106C of the handle.

The shaft 106B of the first portion 106A of the handle 106 may include a ratchet 138. For example, the ratchet 138 may include a series of ridges and/or grooves, which may interact with a feature of the second portion 106C of the handle 106. The ratchet 138 may permit movement of the spool 136 in the distal direction D and may inhibit or prevent movement of the spool 136 in the proximal direction P. Alternatively, the ratchet 138 may permit movement of the spool 136 in both the proximal and the distal directions. The ratchet 138 may provide a stepped control of the motion of the control wire 126, so that a practitioner may better determine and control the movement of the agent delivery member 128. For example, the ratchet may be configured to allow for movement of the control wire 126 in 0.5 mm increments, and may be configured to produce an audible click or tactile feedback to the practitioner, such that the practitioner may determine the positioning of the agent delivery member 128 at the distal end of the control wire 126 by feedback from the audible or tactile feedback of the ratchet 138.

The second portion 106C of the handle 106 may include a lock button 140 or another type of locking mechanism (e.g., slider, knob, lever, etc). Alternatively, the first portion 106A of the handle 106 may include the lock button 140. The first portion 106A of the handle 106 may be locked in place with respect to the second portion 106C of the handle 106 by depressing lock button 140. Locking the first portion 106A of the handle 106 with respect to the second portion 106C of the handle 106 may effectively lock the agent delivery member 128 in place.

After the agent delivery member 128 is positioned at or near the distal end of the insertion portion 108, the practitioner may introduce a treatment agent fluid into the lumen 124 and, thus, the agent delivery member 128 via second medical device 104. In some aspects, the treatment agent fluid is an adhesive (e.g., a prophylactic adhesive) or another type of hemostatic agent. In other aspects, a dried or powdered form of an agent (e.g., cyanoacrylates, albumin and glutaraldehyde, poly (ethylene glycol) (PEG), polyurethane, and fibrin, among other agents) may be embedded (pre-loaded) in the agent delivery member 128 before first medical device 102 is inserted into a scope or other type of insertion device. Then, a fluid such as water, a saline solution, or other fluid may be delivered using the second medical device 104. Upon interacting (e.g., mixing) with the dried or powdered agent, a gel or other type of fluid agent (e.g. a prophylactic agent) may be formed. In any of the above aspects, manipulation of handle 106 may control the deployment, delivery, and positioning of the agent, as discussed in further detail below.

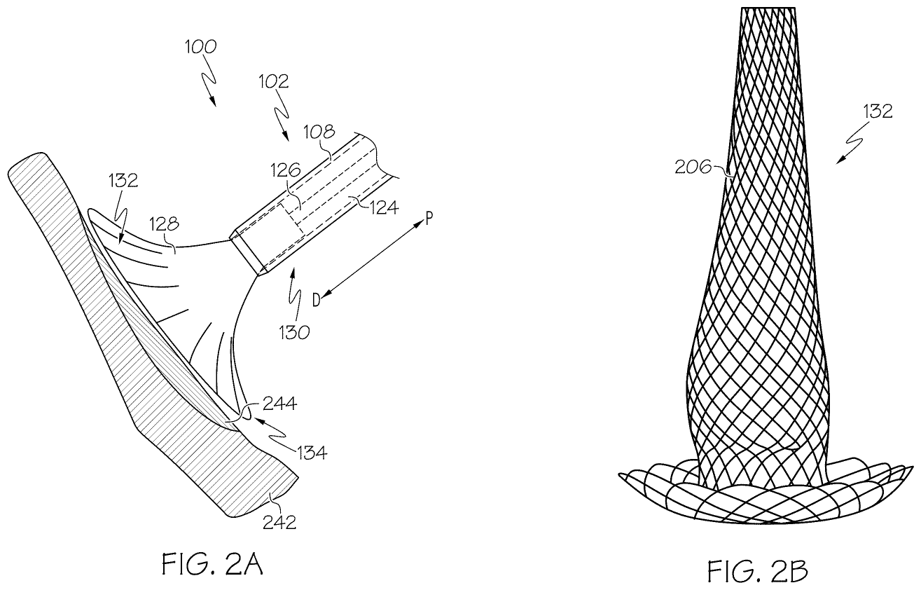

FIG. 2A depicts an aspect of the exemplary medical system 100 in use in an exemplary medical procedure, according to some embodiments. In particular, FIG. 2A depicts the distal end 130 of the insertion portion 108 of the first medical device 102. When used in an exemplary medical procedure, a practitioner may navigate the distal end 130 of the insertion portion 108 to be near or adjacent to a target 244 within a bodily lumen having a wall 242. The target 244 may be, for example, a bleeding ulcer or another type of lesion and the bodily lumen wall 242 may be, for example, the esophagus or stomach of a patient. Once the medical system 100 is in position, the practitioner may actuate the control wire 126 via the spool 136 shown in FIG. 1A (i.e., move the spool 136 distally) to deploy the agent delivery member 128 by moving the agent delivery member 128 distally.

In FIG. 2A, the agent delivery member 128 is shown in an expanded configuration. The expanded configuration of FIG. 2A may be a natural, biased configuration of the agent delivery member 128. Thus, a portion (or an entirety) of the agent delivery member 128 may transition from the collapsed configuration to the expanded configuration when the portion (or the entirety) of the agent delivery member 128 is moved distally of insertion portion 108. In the aspect shown in FIG. 2A, the agent delivery member 128 may bulge out from the distal end 130 of the insertion portion 108 based on a preformed shape of the expandable member 132, which may be formed from a shape memory element such as nitinol. The absorbent member 134 may adopt a similar shape to the expandable member 132, because the expandable member 132 is coupled to the absorbent member 134. For example, as shown in FIG. 2A, the agent delivery member 128 may have a flared shape, such as a curved cone shape. Distal portions of the agent delivery member 128 may have a greater radial diameter/width than proximal portions of the agent delivery member.

As depicted in FIG. 2B, the expandable member 132 may be or include a stent or a similar structure comprised of braided wires 206 (e.g., nitinol wires) with a bulged and inverted shape at a distal end of the expandable member 132. As shown in FIG. 2B, distal edges of expandable member 132 may fold backward, exposing an inner surface of expandable member 132. A shape of expandable member 132 may increase the surface area of the agent delivery member 128 when it is deployed out of the distal end 130 of the insertion portion 108.

The absorbent member 134 may be composed of a foam or sponge material that is formed over the expandable member 132, such that when the expandable member 132 expands due to its preformed shape upon being released from the insertion portion 108, the absorbent member 134 likewise expands. In some examples, the absorbent member 134 may be on a radially inner surface of the expandable member 132, such that the absorbent member 134 contacts the target 244 when the delivery member 128 is pressed against or otherwise contacts the target 244. In other examples, the absorbent material 134 may be on a radially outer surface of the expandable member 132, and the fluid contained within the absorbent material 134 may pass through openings in the expandable member 132 when the delivery member 128 is pressed against or otherwise contacts the target 244. Thus, with reference back to FIG. 2A, as the agent delivery member 128 is pressed against the target 244 within the bodily lumen wall 242, the force produced by the pressure of the agent delivery member 128 against the target 244 and capillary action within the absorbent member 134 deliver the treatment agent fluid to the target 244.

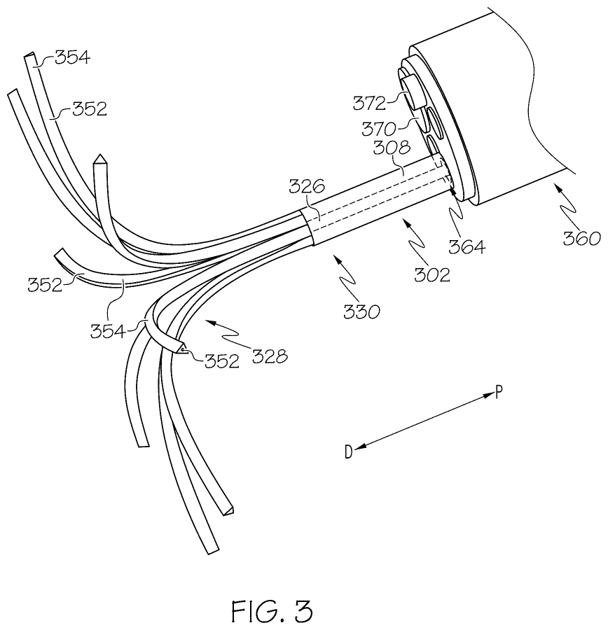

FIG. 3 shows a distal end 330 of an insertion portion 308 of an alternative first medical device 302. In the alterative aspect shown in FIG. 3, the expandable member of an agent delivery member 328 includes a plurality of discrete wires 352 (or struts) with a preformed, flared, curved shape, with each wire 352 being embedded within or covered by a respective absorbent covering 354. For example, each wire 352 may flare away from a central longitudinal axis of insertion portion 308. Together, the wires 352 may form an overall curved conical shape of the agent delivery member 328.

Thus, instead of the single absorbent member 134 of first medical device 102, the first medical device 302 shown in FIG. 3 comprises a plurality of absorbent coverings 354, with each absorbent covering 354 corresponding to and at least partially covering (e.g., enveloping) a respective expandable member wire 352. The wires 352 may be formed of a memory shape material such as nitinol. The expandable member may be formed of a plurality of discrete member wires 352 that are biased to a flared shape in the expanded configuration. The discrete member wires 352 are not connected to each other by a membrane or other material.

Similar to the aspect shown in FIGS. 2A-2C, the agent delivery member 328 is actuated by control wire 326 to be pressed against a target within a bodily lumen, and the force produced by the pressure of the agent delivery member 328 against the target and capillary action of the absorbent coverings 354 deliver the treatment agent fluid to the target via each of the plurality of wires 352 within the agent delivery member 328.

Also depicted in FIG. 3, the first medical device 302 may be inserted into an insertion device 360, such as a gastroscope or one of other similar insertion devices mentioned above. The insertion device 360 may include a working channel with a distal opening 364 at a distal portion of the insertion device 360. Insertion device 360 may include one or more of: one or more illumination device(s) 370 (e.g., one or more LEDs, optical fibers, etc.), and one or more imaging device(s) 372 (e.g., one or more cameras). Although insertion device 360 is depicted only in FIG. 3, it will be appreciated that any of the examples disclosed herein may be utilized with insertion device 360.

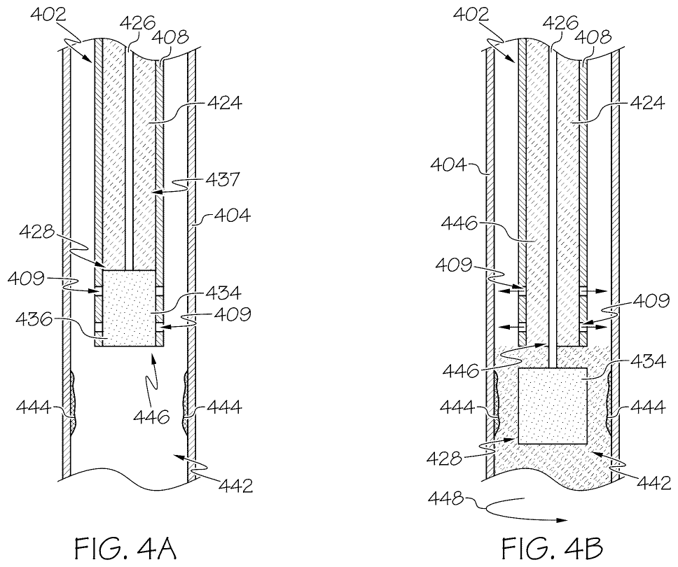

FIGS. 4A and 4B depict an alternative first medical device 402. In some aspects, an insertion portion 408 of first medical device 402 may include an agent delivery member 428 configured to expand radially outward after it is deployed out of (moved distally of) the distal end of the insertion portion 408. FIG. 4A depicts an the agent delivery member 428 in a first, collapsed or restrained configuration within the insertion portion 408, the shaft having been navigated by a practitioner to a target 444 within a bodily lumen 442 of a patient. The agent delivery member 428 may include an absorbent member 434. For example, the absorbent member 434 may be a cylindrically shaped sponge. In some examples, the agent delivery member 428 may include expandable member (not shown) within the absorbent member 434, which may be biased to bulge outward in a radial direction. Alternatively, the expandable member may be omitted, and the absorbent member 434 may be expandable in a radial direction upon wetting of the absorbent member 434.

In some examples, the agent delivery member 428 may be pre-loaded with one or more powdered hemostatic agents 436. The powdered hemostatic agents 436 may include, for example, cyanoacrylates, albumin and glutaraldehyde, poly (ethylene glycol) (PEG), polyurethane, and fibrin, among other agents. In a delivery configuration of the first medical device 402, the agent delivery member 428 may be constrained within and near a distal end of a lumen 424 extending through the insertion portion 408. The lumen 424 may be in fluid communication with the second medical device 104 via the handle 106, as discussed above with respect to the embodiment of FIGS. 1A and 1B.

A control wire 426 (having any of the properties of the control wire 126) coupled to the agent delivery member 428. As shown in FIG. 4B, the control wire 426 may be moved distally in order to move the agent delivery member 428 distally, pas a distalmost end of the insertion portion 408. When no longer constrained by surfaces of the lumen 424, the agent delivery member 428 may be free to expand radially outward to a biased, expanded configuration. Thus, the agent delivery member 428 may transition from the collapsed configuration to the expanded configuration. In the expanded configuration, the agent delivery member 428 may be pressed against the target 444 formed on the walls 404 of the bodily lumen 442, as shown in FIG. 4B. The control wire 426 is actuated by the practitioner to deploy the agent delivery member 428, and the lumen 424 allows for the delivery of a fluid 437 to the agent delivery member 428.

The fluid 437 (e.g., water, saline, an active agent, air, or any other suitable fluid) may be introduced into the lumen 424 before or after the agent delivery member 428 is deployed distally of the insertion portion 408. For example, prior to deployment of the agent delivery member 428, the agent delivery member 428 may obstruct a distal opening 446 of the lumen 424. Furthermore, the insertion portion 408 may have one or more radially outer openings 409 formed in radially outer walls of insertion portion 408. The radially outer openings 409 may be blocked by agent delivery member 428 (e.g., distal to a proximalmost end of agent delivery member 428) in a configuration in which agent delivery member 428 is disposed within lumen 424.

After the agent delivery member 428 is deployed (moved distally out of distal opening 446), fluid may flow through the distal opening 446 and radially outer openings 409 (as shown by the arrows in FIG. 4B). The fluid may interact with (e.g., mix with) and activate the agent 436 on agent delivery member 428, as discussed above. In some aspects, the fluid delivered from the fluid delivery device 104 via the lumen 424 may be a saline fluid or air that, rather than interacting with the agent delivery member 428 to produce a hemostatic gel, is used to deploy (e.g., spread) the agent delivery member 428. The agent delivery member 428 may transition to the expanded configuration (e.g., expand radially outward), to allow for increased contact with target 444.

Once deployed, a practitioner may rotate an entirety of the handle 106 or an actuator of the handle 106 in order to rotate the agent delivery member 428 about a central longitudinal axis of the insertion portion 108. This rotation may serve to increase contact with the target 444 and/or provide a greater area of contact with the walls 404 of the bodily lumen 442 and/or to help to provide uniform distribution of the agent activated agent 436.

Multiple agents may be added in a mixture or serially in layers, for example, each from a respective fluid delivery device 104. Cross linking between layers may provide a therapeutic advantage, such as preventing bleeding, closing perforations, and aiding in drug delivery.

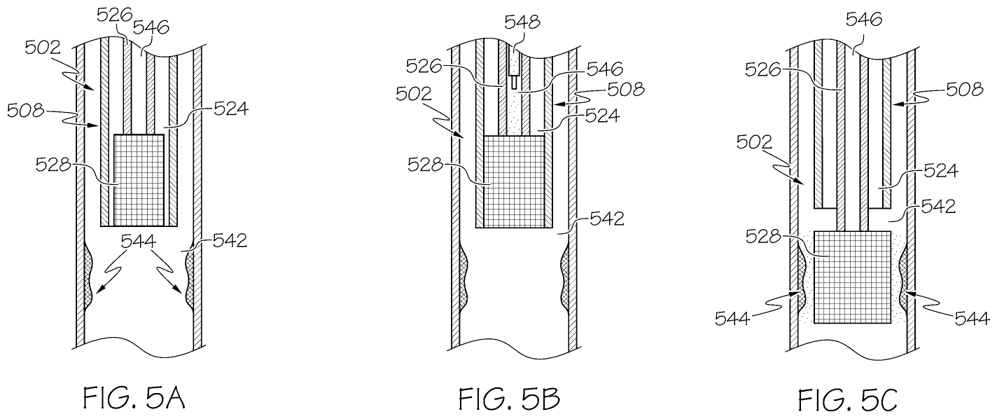

FIGS. 5A-5C depict an exemplary medical device according to yet other aspects. The first medical device 502 depicted in FIG. 5A includes an agent delivery member 528 connected to an actuation member 526, both disposed in a lumen 524 of an insertion portion 508. The agent delivery member 528 may include a capsule or container. In some examples, the agent delivery member 528 may include an absorbent and/or expandable member having any of the properties of the absorbent members and expandable members described above. For example, the agent delivery member 528 may include a cylindrical sponge, foam, or other porous body. The agent delivery member 528 may house or may be embedded, impregnated, coated, or otherwise treated with a powdered or other dry form of one or more agents, such as hemostatic agents including, for example, cyanoacrylates, albumin and glutaraldehyde, poly (ethylene glycol) (PEG), polyurethane, and fibrin, among other agents.

In the aspects depicted in FIGS. 5A-5C, actuation member 526 may include a hollow interior 546 that defines a lumen. For example, the actuation member 526 may include a tube. As such, a fluid may be delivered through the hollow interior 546 of the actuation member 526.

FIG. 5A depicts a first configuration of first medical device 502, in which the agent delivery member 528 may have a collapsed configuration. The agent delivery member 528 may be constrained in the collapsed configuration by walls of the insertion portion 508. In the first configuration, the agent delivery member 528 may be entirely received within the lumen 524, such that a distal end of the agent delivery member is proximal of a distalmost end of the insertion portion 508. Although agent delivery member 528 is described herein as having a collapsed configuration and an expanded configuration, it will be appreciated that agent delivery member 528 may lack such expanded and collapsed configurations and may have a uniform size in all configurations.

FIG. 5B depicts a second configuration of the first medical device 502, in which a fluid may be delivered to the agent delivery member 528. The fluid may be, for example, a saline fluid, and may combine with the powdered or otherwise dry agent(s) of the agent delivery member 528 to form a viscous treatment gel or another treatment fluid. For example, a catheter 548 may be inserted into the hollow interior 546 of the actuation member 526. The hollow interior 546 may be in fluid communication with the agent delivery member 528, such that the fluid delivered from the catheter 548 may contact and disperse within the agent delivery member 528.

As shown in FIG. 5C the practitioner may then deploy the agent delivery member 528 from the distal end of the insertion portion 508 via the actuation member 526. For example, the actuation member 526 may be moved distally. Fluid and/or air pressure may be applied via the hollow interior 546 to provide kinetic energy to the agent delivery member 528 and the agent(s) provided in the agent delivery member 528 for application to the target tissue 544. The agent delivery member 528 may be biased to an expanded configuration, such that it expands radially outward when no longer constrained by the insertion portion 508. Thus, the agent delivery member 528 may transition from the collapsed configuration to the expanded configuration. In alternatives, the agent delivery member 528 may have the same size in the configurations of FIGS. 5A-5C and may not expand. In the example shown in FIG. 5C, the agent delivery member may expand radially to contact target 544 within bodily lumen 542. However, the concepts described in this aspect may be applied to any of the aspects previously described.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A medical device, comprising:
a handle;
a shaft extending from a distal end of the handle, the shaft defining a lumen;
a pull wire disposed in the lumen of the shaft;
an agent delivery member connected to a distal end of the pull wire, the agent delivery member comprising an expandable member and an absorbent member, wherein the expandable member is configured to transition from a collapsed configuration to an expanded configuration, and wherein the expandable member is biased to the expanded configuration.

2. The medical device of claim 1, wherein the lumen is configured to transmit a fluid from the handle of the device to the agent delivery member.

3. The medical device of claim 1, wherein the absorbent member includes a powdered treatment agent.

4. The medical device of claim 3, wherein the powdered treatment agent is configured to mix with a fluid delivered through the lumen to form a viscous fluid treatment gel.

5. The medical device of claim 1, wherein, in the expanded configuration, the agent delivery member has a curved cone shape.

6. The medical device of claim 1, wherein the expandable member includes a memory shape element.

7. The medical device of claim 6, wherein the expandable member includes nitinol.

8. The medical device of claim 6, wherein the expandable member includes braided nitinol wires.

9. The medical device of claim 6, wherein the expandable member is formed of a plurality of discrete wires that are biased to a flared shape in the expanded configuration.

10. The medical device of claim 9, wherein each of the plurality of wires includes an absorbent member.

11. The medical device of claim 1, wherein the expandable member is configured to expand radially outward when the agent delivery member is deployed from the distal end of the shaft.

12. The medical device of claim 1, wherein the absorbent member includes a sponge.

13. The medical device of claim 1, wherein the pull wire defines a pull wire lumen.

14. The medical device of claim 1, wherein pull wire lumen is configured to transmit a fluid to the absorbent member.

15. The medical device of claim 1, wherein the absorbent member is on a radially inner surface of the expandable member.

16. A medical device, comprising:
a handle;
a shaft extending from a distal end of the handle, the shaft defining a lumen;
a pull wire connected to the handle; and
an agent delivery member connected to a distal end of the pull wire, the agent delivery member comprising an absorbent member;
wherein the shaft includes a lumen for delivering fluid to the absorbent member.

17. The medical device of claim 16, wherein the lumen for delivering fluid to the absorbent member is disposed in the pull wire.

18. A method of using a medical device, the medical device comprising an agent delivery member connected to a distal end of a pull wire, the agent delivery member comprising an expandable member and an absorbent member, method comprising:
delivering fluid to the absorbent member of the agent delivery member;
moving the agent delivery member distally so that it transitions from a collapsed configuration to an expanded configuration; and
using the agent delivery member to apply an agent to a bodily lumen.

19. The method of claim 18, wherein the absorbent member includes a powdered treatment agent configured to mix with the fluid to form a viscous fluid treatment gel.

20. The method of claim 18, wherein the expandable member includes a shape memory element, and, in the expanded configuration, the agent delivery member has a curved cone shape.
